(19) United States
(12) Patent Application Publication
(10) Pub. No.: US 2012/0129770 A1
(43) Pub. Date: May 24, 2012

(54) NOVEL POLYNUCLEOTIDE MOLECULES FOR ENHANCED GENE EXPRESSION

(76) Inventors: Rajyashri Karur Ramakrishna, Hubli (IN); Ashok Kumar, Mumbai (IN); Annapoorni Jegatheesan, Hubli (IN); Jonnala Ujwal Kumar, Hubli (IN); Veeresh Sangappa Hugar, Hubli (IN)

(21) Appl. No.: 13/380,394

(22) PCT Filed: Jun. 22, 2010
(86) PCT No.: PCT/IN10/00429

§ 371 Date: Dec. 22, 2011

(30) Foreign Application Priority Data
Jun. 22, 2009 (IN) 1478/MUM/2009

(51) Int. Cl. A61K 38/18; C07K 14/505; C12P 21/00; C12N 15/85; C12N 5/10; C12N 15/12; A61P 7/00

(52) U.S. Cl. 514/7.7; 536/23.5; 530/380; 435/320.1; 435/358; 435/69.6

(57) ABSTRACT

Disclosed herein is an isolated polynucleotide molecule encoding protein such as erythropoietin or structural variants, comprising an expression regulating nucleotide sequence operatively linked to a nucleotide sequence(s) encoding said proteins. The said expression regulatory region comprises of Exon A and at least proximal region of Intron A of Major Immediate Early Region of human Cytomegalovirus or its functional variants which when transfected into host cells results in many fold increase in expression of the protein.

### FIELD OF INVENTION

Present invention relates to polynucleotide molecules encoding proteins such as erythropoietin or structural variants, comprises an expression regulating nucleotide sequence operatively linked to a nucleotide sequence(s) encoding said proteins. The invention further relates to expression vectors for transfection in host cells expressing Erythropoietin or its structural variants, and host cells transfected with the novel polynucleotide molecules. The invention also relates to use of novel expression vectors or host cells containing polynucleotide molecules of the present invention for preparing recombinant protein such as Erythropoietin(s) or structural variants including its isoform(s) and pharmaceutical compositions made thereof. The present invention is characterized by higher protein expressions with the use of polynucleotide molecules of the present invention.

### BACKGROUND OF THE INVENTION

Erythropoietin (EPO) is a mammalian glycoprotein hormone that is the primary regulator of erythropoiesis or maintenance of the body's red blood cell mass at an optimum level. Erythropoietin is mainly produced in the kidney and liver in response to low oxygen levels. The secreted hormone bind specific receptors on the surface of red blood cell precursors in the bone marrow, leading to their survival, proliferation, differentiation and ultimately to increase in the haematocrit (i.e., the ratio of the volume occupied by packed red blood cells to the volume of the whole blood).

Erythropoietin and its function was first discovered by Dr. John Adamson and Dr. Joseph W. Eschbach in 1970s, and proposed as a treatment option for anemia in humans. Subsequently, it has been established that this hormone regulates RBC production and also has other important vital function like its role in wound healing including neuronal injury. Accordingly numerous pharmaceutical applications were proposed and studied for erythropoietin and its structural variants.

Naturally occurring human erythropoietin is first translated to a 166 amino acid containing polypeptide chain with arginine at position 166. In a posttranslational modification, arginine 166 is found to be cleaved by a carboxypeptidase. The primary structure of 165 amino acid human erythropoietin can be found in Lin et. al. (1985) Proc. Natl. Acad. Sci. Vol. 82, pp. 7580-7584, which may be accompanied by a leader sequence. The secondary structure of erythropoietin includes two disulfide bridges between Cys7-Cys161 and Cys29-Cys33. Fully glycosylated EPO comprises approximately 40% carbohydrate groups by molecular weight (Sasaki, H., et al., J. Biol. Chem. 262 (1987) 12059-12076).

Since its introduction more than a decade ago, recombinant human EPO (rHuEPO) has become the standard of care in treating anemia associated with chronic renal failure (CRF). It is highly effective in correcting the anemia, restoring energy levels, and increasing patient well being and quality of life. It has also been approved for the treatment of anemia associated with cancer, HIV infection, and use in surgical setting to decrease the need for allogenic blood transfusions.

Subsequent to discovery of wider application of erythropoietin, attempts were made to improve the efficiency or activity of erythropoietin and thereby many structural variants or isoforms were discovered. One such discovery, published in WO9105867 & WO9505465 discloses the erythropoietin derivatives with at least one amino acid change in the primary amino acid structure of erythropoietin, which eventually resulted in increased sialic acid content in the protein made thereof. This research resulted in new generation erythropoietin isoforms or structural variants exhibiting higher plasma stability and improved half-life of the product during human application. Darbepoetin alpha is the first such erythropoietin derivative approved by FDA and sold under the trade name of Aranesp.

There is a growing demand for biologicals which are primarily due to high specificity of these medicines to disease targets which results in significantly reduced and well defined risk of toxicity compared to small chemical molecules/drugs. Further, by employing recombinant techniques to produce these biologicals in contrast with older techniques of purifying them from tissue extracts or body fluids, products of very high purity, and well-defined safety and physicochemical characteristics, can be easily produced. Despite having these superior qualities in pharmaceutical application, most recombinant biologics remain inaccessible to common people because they continue to remain prohibitively expensive.

Many factors normally control the amount of product produced by a host cell, including factors which are external to the host cell such as the culturing conditions that may be optimized to maximum production levels by trial. But there are factors internal to the cells, majority of them include factors that regulate the efficiency and quality of transcription, and translation in the host cell systems, and are predominantly dependent upon the design of the expression vector. There are many attempts reported in literature to increase the production levels of proteins like erythropoietin by improving either the culturing conditions or construction of vectors adapted to express increased levels of erythropoietin.

Most of these attempts were made to change internal factors for increasing the production yield of Erythropoietin. The internal factors regulate gene expression in many ways, which are not fully characterized but may be specific to host cells and the target protein. Exemplary literature include the following: EP1428878 discloses production of erythropoietin with the use of a DNA vector construct comprising a CMV promoter, DHFR selection in CHO cells operatively linked to the nucleotide encoding erythropoietin and reported an expression level of 1800 IU/ml (45 mg/l) of erythropoietin. Stinski et al in U.S. Pat. No. 5,385,839 discloses utilization of the CMV promoter attached with immediate early promoter region of hCMV having over 403 base pair sequence with specific repeat units and their use in expression vectors for increased expression. Human cytomegalovirus major immediate early region (herein after referred as MIER) has been reported by Hennighausen in EMBO (1986) vol. 5 no. 6 pp. 1367-1371, which remains in focus for modulating the expression levels of genes in subsequent literature. Ghazal et. al. in Journal of Virology, (May 1991), p. 2299-2307, thus, identified cytomegalovirus MIER exon A, and concluded the significance of 18 bp conserved box in providing better expression levels of proteins. However, Chapman et. al. Nucleic Acids Research, (1991) Vol. 19, No. 14 3979-3986, reported reduced level of expression with the use of hCMV major immediate early region in the absence of complete hCMV intron A. and also indicated that expression levels are gene specific in obtaining high glycoprotein expression. Chapman, therefore, suggested a heterologous vector with intron A for development. These findings were further validated by R. D. Simari in Molecular Medicine (1998) vol 4: 700-706, in which MIER is experimented to see effects of individual regions of Exon A & Intron A in expression by way of deletions of specific portion(s), however, established that entire HCMV MIER with exon A & intron A provides highest expression among all alternatives and in some experiments this had shown 7 folds expression in comparison to intron A truncated versions and intron A deficient versions. Simari et al. reported that increased expression may also depend on molecular weight of mRNA having large/major portion of intron A.

The use of another widely used promoter was disclosed by Lin Fu in U.S. Pat. No. 5,955,422 for production of erythropoietin wherein an SV40 promoter attached with sequence encoding erythropoietin with poly adenylation sequence and DHFR was used for transfection in CHO cells and reported to get expression levels of 750-1470 IU/10⁶ cells/48 hrs; Similarly an EF1 promoter was utilized to increase the production of EPO, Delcuve in U.S. Pat. No. 5,888,774 discloses use of the EF1 promoter in combination with apolipoprotein & SAR elements and claims to produce EPO, with expression of 1500-1700 IU/10⁶ cells in 24 hrs. Furthermore, EP0255231 reported an expression of 7000 IU/ml in COS7 (Monkey Kidney) type of cell using an expression vector containing adenovirus2 major late promoter and MT-I promoter. EP205564 reported expression levels reaching up to 180 ng/ml using MMT promoter in COS cells. Recently Arun et. al. in WO2007017903 reported production levels of 2366 IU/ml/24 hr erythropoietin in the CHO-DHFR cell lines using a CMV promoter characterized by linking it to Adenovirus tripartite leader element (TPL) and a hybrid (chimeric) intron. Shabnam et. al. in WO2004089973 reported the expression of protein 165-211 IU/ml/day in HEK 293 cell lines, and Yallop, US2006121611, provided way to produce 1600 IU/ml/day (40/mg/l/day) expression in PER.C6 cells.

Regardless of afore-mentioned advances, the high cost of manufacturing of recombinant proteins, especially those utilizing mammalian expression systems, still remains a major concern mainly due to larger variations in the production levels, and mostly these limitations are specific to individual proteins and expression systems used thereof.

Therefore, life saving drugs like erythropoietin and its structural variants like Darbepoetin are affordable only to a small fraction of population affected with life threatening disease like anemia while a vast majority cannot use them enough. Thus there is an urgent need to bring down the cost of these biological drugs.

A serious limitation to reduce cost is associated with manufacturing of recombinant proteins and expression capability of existing strains.

The elucidation to the same is the development of expression vectors or DNA constructs that would give high level of protein expression still requires empirically testing of many possibilities, though the success is limited by various factors. Therefore, even though prior art reports various methods for protein expression by modulating the expression vector, it is still desirable to develop new expression systems for increasing the productivity of eukaryotic host cells and for reproducible production of highly valuable proteins of interest.

In lieu of above it is the object of the present inventor to invent new DNA molecules or expression system adaptable to host cells giving higher expression yield of protein of interest, mainly erythropoietin or its isoforms/structural variants.

In other words, it is an object of the present invention to provide recombinant polynucleotide molecules encoding higher expression levels of erythropoietin or its structural variants.

### SUMMARY OF THE INVENTION

Accordingly, the present invention provides isolated polynucleotide molecules encoding erythropoietin or its structural variants, comprising of an expression regulating nucleotide region operatively linked to a nucleotide sequence(s) encoding the subject proteins. The expression regulatory region comprises of Exon A and at least proximal region of Intron A (herein after it is referred as IEax) of Major Immediate Early Region (MIER) of human Cytomegalovirus (herein after it is referred as hCMV) or its functional variants. Expression vectors inserted with polynucleotide molecule of the present invention, when transfected into host cells surprisingly resulted in many fold increase in expression of protein compared to reports with conventional vector for erythropoietin, or its isoform.

Accordingly, the present invention provides an isolated polynucleotide molecule comprising an expression regulating polynucleotide sequence IEax (sequence ID 1) operatively linked to nucleic acid sequence encoding a recombinant protein such as erythropoietin or structural variants thereof. In a preferred embodiment, the polynucleotide molecule contain nucleotide sequences encoding erythropoietin as represented in SEQ ID No 4 or darbepoetin as represented in SEQ ID No. 5. More preferably, the region linking the IEax to sequence encoding protein of interest referred as linker may comprise further introns; exons; enhancers; primers; restriction enzyme sites for manipulation, analysis or further increase of expression; more preferably the linker may comprise a short polynucleotide sequence herein after referred as “IPNS” defined by the sequence ID 2 (FIG. 3), or its functional variants. Polynucleotide joining promoter and gene of desired protein comprises about 315 bp nucleotide, and/or it is operatively attached to erythropoietin or darbepoetin gene.

In another aspect, the present invention provides expression vectors for transfection in host cells comprising the polynucleotide molecule of the present invention, wherein the polynucleotide molecule comprises MIER sequence IEax referred herein before or its functional variants operatively linked to the nucleotide of erythropoietin or its structural variants and regulates the level of expression of resulting protein in the host cell.

In a further aspect, the present invention provides stable host cell systems transfected with the polynucleotide molecule or expression vector comprising polynucleotide sequence, having the regulatory region lEax, or its functional variants operatively linked to the nucleotide encoding protein such as erythropoietin or its structural variants. The host cell system includes, but not limited to, any eukaryotic host cell preferably mammalian cell adaptable to express the subject protein of the invention. A preferred embodiment includes mammalian host cell transfected with the expression vector comprising the polynucleotide construct of sequence ID 8 encoding darbepoetin (sequence ID 5) or the polynucleotide construct of sequence ID 9 encoding erythropoietin (sequence ID 4). The host cells of the present invention are adapted to express recombinant erythropoietin or its isoforms at higher levels, at least 500 IU/ml/24 hours, preferably over 3,200 IU/ml/24 hours, preferably over 32,000-96,000 IU/ml in three days. This invention may also be applied for large scale production of other therapeutically active proteins of mass use, which is expected to give a production rate of over 3200 IU/ml/24 hrs or much more.

In a further aspect, the invention provides a method to produce high levels of erythropoietin or its structural variants comprising culturing expression vectors and the host cells of the present invention in a suitable harvesting medium until sufficient amounts of erythropoietin are produced by the cell and separating the erythropoietin or structural variants thus obtained.

In one aspect the present invention provides erythropoietin produced by the following process steps:
a. culturing a host cell transfected with an expression vector containing a expression regulating region IEax operatively linked to nucleotide sequence encoding erythropoietin in a suitable medium for sufficient period;
b. separating erythropoietin from the cell and culturing medium in step (a); and
c. purifying erythropoietin.

In another aspect the present invention provides Darbepoetin produced by the following process steps:
a. culturing a host cell transfected with an expression vector containing a expression regulating region IEax operatively linked to nucleotide sequence encoding Darbepoetin in a suitable medium for sufficient period;
b. separating Darbepoetin from the cell and Culturing medium in step (a); and
c. purifying Darbepoetin.

In a further aspect, the invention also provides pharmaceutical compositions comprising the erythropoietin or darbepoetin or its structural variants obtained with the use of polynucleotide molecule, expression vector, and host cell systems of the present invention. In one embodiment, the present invention provides a method of treating a patient with the polynucleotide molecule of the present invention encoding human erythropoietin or recombinant darbepoetin in the mammalian cell system to accomplish a curative action in the body.

In yet another aspect, the invention provides use of polynucleotide molecule of the present invention encoding human erythropoietin or recombinant darbepoetin in the mammalian cell system in a medicament for curative action in the body.

The details of one or more embodiments in the practice of the inventions are set forth in the description below. Other features, objects and advantages of the inventions will be apparent from the appended examples and claims.

### BRIEF DESCRIPTION OF THE FIGURES

FIG. 1. Map of the isolated polynucleotide of the present invention.

FIG. 2. Map of the isolated polynucleotide of the present invention with details of linker

FIG. 3. Sequence of IPNS (SEQ. ID. 2)

FIG. 4. Sequence of IEax, according to the present invention. (SEQ. ID. 1)

FIG. 5. Sequence of Erythropoietin (SEQ. ID. 5)

FIG. 6. Sequence of Darbepoetin (SEQ. ID. 4)

FIG. 7. Exemplary polynucleotide of Darbepoetin for enhanced gene expression according to present invention.

FIG. 8. Exemplary polynucleotide of Erythropoietin for enhanced gene expression according to present invention.

FIG. 9. Map of Expression Vector with conventional polynucleotide of Darbepoetin gene

FIG. 10. Map of Expression Vector with polynucleotide of Darbepoetin gene for enhanced expression according to present invention.

FIG. 11. Sequence of IPNS and IEax, according to the present invention (SEQ. ID. 3)

FIG. 12. Exemplary polynucleotide sequence according to invention (SEQ. ID. 8)

FIG. 13. SDS Page comparison of product by Invention (A) in two different concentrations (50 μg and 5 μg) and Standard Darbepoetin (Std) (50 μg)

FIG. 14 Isoelectric focusing gel (IEF) comparison between product by invention (A) and Standard Darbepoetin (S).

### DETAILED DESCRIPTION

Unless specified otherwise, all technical and scientific terms used herein have the same meaning as commonly understood by one of ordinary skill in the art, to which this invention belongs. Although any methods and materials similar or equivalent to those described herein can be used in the practice or testing of the present invention, the preferred methods and materials are described. To describe the invention, certain terms are defined herein specifically as follows.

Unless stated to the contrary, any of the words “having” “including,” “includes,” “comprising,” and “comprises” mean “including without limitation” and shall not be construed to limit any general statement that it follows to the specific or similar items or matters immediately following it. Embodiments of the invention are not mutually exclusive, but may be implemented in various combinations. The described embodiments of the invention and the disclosed examples are given for the purpose of illustration rather than limitation of the invention as set forth the appended claims.

Unless stated to the contrary “erythropoietin” means biologically active erythropoietin including naturally occurring erythropoietin, urinary derived human erythropoietin as well as non-naturally occurring polypeptides having an amino acid sequence and glycosylation sufficiently duplicative of that of naturally occurring erythropoietin to allow possession of in vivo biological properties causing bone marrow cells to increase production of reticulocytes and red blood cells or a recombinant erythropoietin having similar properties. Erythropoietin of the present invention includes all its isoforms. “Isoforms” as used herein means structural modifications in the protein other than changes in the primary amino acid, which may or may not affect the biological activity of the recombinant erythropoietin, which encompasses epoetin alfa, Procrit, Eprex, Epogen, epoetin beta and the like. “structural variants or derivatives of erythropoietin” as used herein means erythropoietin having at least one amino acid change in the primary amino acid structure of erythropoietin, which encompasses darbepoetin.

The term “recombinant” when used with reference to a cell typically indicates that the cell replicates a heterologous nucleic acid or expresses a polypeptide encoded by a heterologous nucleic acid. Recombinant cells can comprise genes that are not found within the native (non-recombinant) form of the cell. Recombinant cells also include those that comprise genes that are found in the native form of the cell, but are modified and reintroduced into the cell by artificial means. Recombinant DNA technology includes techniques for the production of recombinant DNA in vitro and transfer of the recombinant DNA into cells where it may be expressed or propagated, thereby producing a recombinant polypeptide.

The term “recombinant expression cassette” or simply an “expression cassette” is a nucleic acid construct or molecule, generated recombinantly or synthetically, with nucleic acid elements that are capable of effecting expression of a structural gene in hosts compatible with such sequences.

The term “encoding” refers to the ability of a nucleotide sequence to code for one or more amino acids. An amino acid sequence can be encoded in any one of six different reading frames provided by a polynucleotide sequence and its complement.

The term “control sequence” is defined herein to include all components, which are necessary or advantageous for the expression of a polypeptide of the present invention. Each control sequence may be native or foreign to the nucleotide sequence encoding the polypeptide. Such control sequences include, but are not limited to, a leader, polyadenylation sequence, propeptide sequence, promoter, signal peptide sequence, and transcription terminator. At a minimum, a control sequence includes a promoter, and transcriptional and translational stop signals. The control sequences may be provided with linkers for the purpose of introducing specific restriction sites facilitating ligation of the control sequences with the coding region of the nucleotide sequence encoding a polypeptide.

The term “coding sequence” refers to a nucleotide sequence that directly specifies the amino acid sequence of its protein product. The boundaries of the coding sequence are generally determined by an open reading frame (ORF), which may begin with the ATG start codon.

The term “MIER”, wherever referred to indicate Major immediate early region of human cytomegalovirus. The term “IEax” wherever referred to means an expression regulatory region comprised of Exon A and at least proximal region of Intron A of major immediate early region (MIER) of human cytomegalovirus or its functional variants.

The present inventors have invented an ‘isolated polynucleotide molecule’ and expression vectors containing them, which when provided in suitable host cell surprisingly found to yield significantly higher levels of protein(s) of interest and thereby reduces the high cost of manufacturing proteins. It has been found that an isolated polynucleotide molecule made by linking an expression regulatory region comprised of Exon A and at least proximal region of Intron A of major immediate early region (MIER) of human cytomegalovirus or its functional variants (lEax sequence) upstream to the nucleotides encoding subject biologic protein surprisingly produces many fold high levels of erythropoietin or its structural variants compared to DNA constructs commonly used in the presence of accessory elements of vector (FIG. 1). The proximal region of Intron A of major immediate early region is especially the sequence starting from 1265 and ending at 1316 characterized by first restriction site of SphI as present in Chapman et. al. Nucleic Acids Research, (1991) Vol. 19, No. 14 3979-3986.

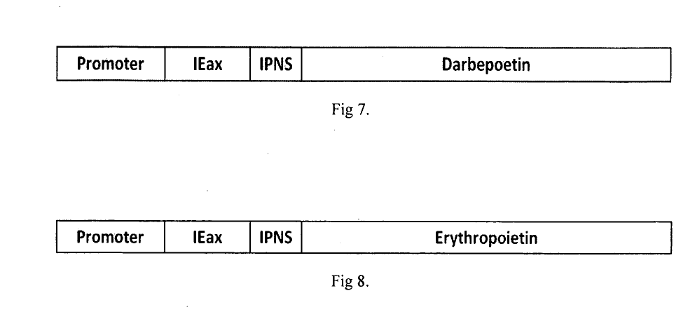

Thus, the present invention provides recombinant polynucleotide molecules encoding erythropoietin or its structural variants, comprises an expression regulating region IEax sequence operatively linked to the nucleotide sequence encoding the subject protein (FIGS. 7, 8). IEax combined with IPNS comprise sequence length about 315 bp.

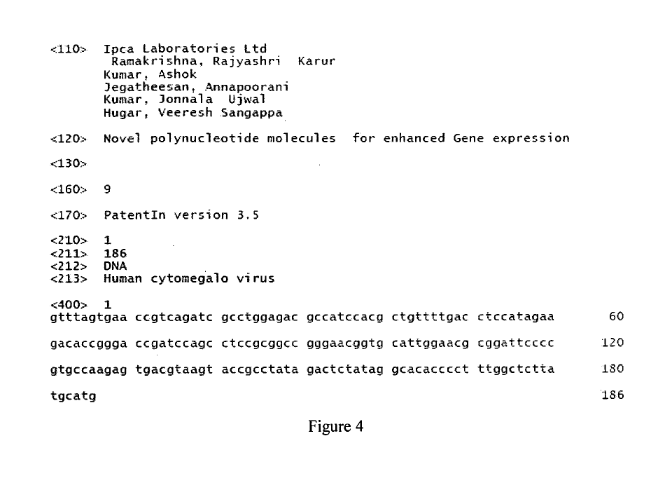

The expression regulating polynucleotide IEax sequence is of major immediate early exon A and proximal intron A and is derived from the regulatory region of the human cytomegalovirus (hCMV). The invention also includes the functional variants of present IEax sequence for regulation of expression in cells (FIG. 4; SEQ ID 1).

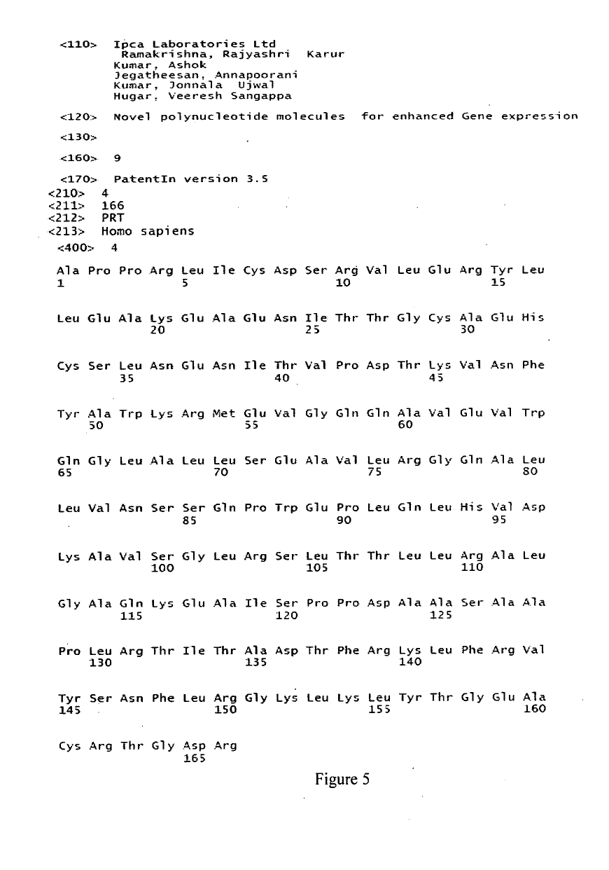

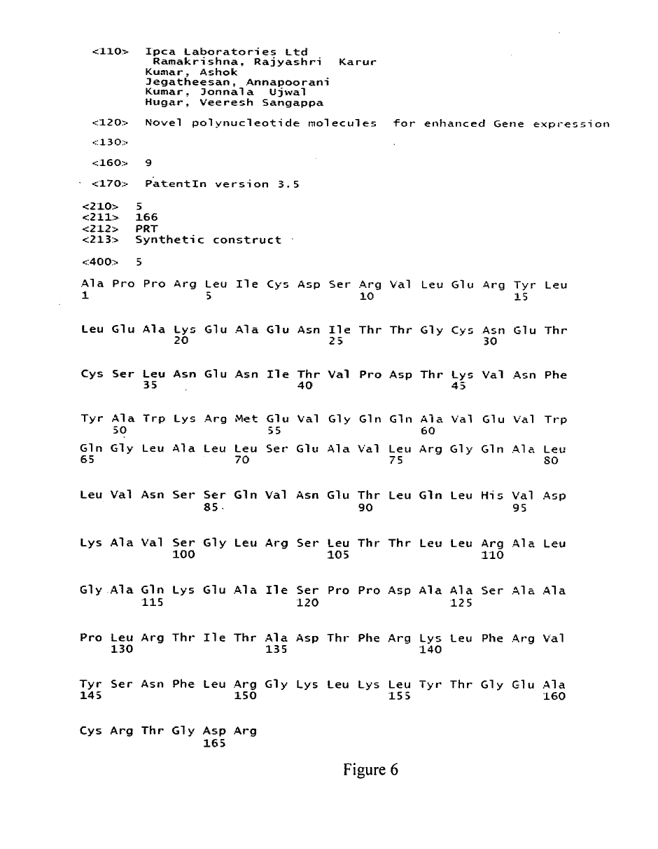

“a nucleotide encoding erythropoietin or its structural variants” includes any nucleic acid molecule which can encode protein having primary structure of erythropoietin (FIG. 5; SEQ ID 4), or its structural variants or derivative of erythropoietin. The preferred nucletotide sequence according to the invention includes sequence codes for epoetin alfa, Procrit, Eprex, Epogen, epoetin beta and the like and structural variants especially darbepoetin (FIG. 6; SEQ ID 5). Preferably, the nucleotide sequence contains codes for the leader sequence of the polypeptide, which may permit secretion of the above proteins from a cell transfected with the recombinant DNA molecule of the invention. The term also encompasses cells that comprise a nucleic acid endogenous to the cell that has been modified without removing the nucleic acid from the cell; such modifications include those obtained by gene replacement, site-specific mutation, and related techniques known to those of ordinary skilled in the art. Erythropoietin in biological system first translated to a 166 amino acid containing polypeptide chain with arginine at position 166. In a postranslational modification, arginine 166 is cleaved by a carboxypeptidase to leave 165 amino acids. Therefore optionally polynucleotide molecule could have sequence wherein last amino acid codon is deleted.

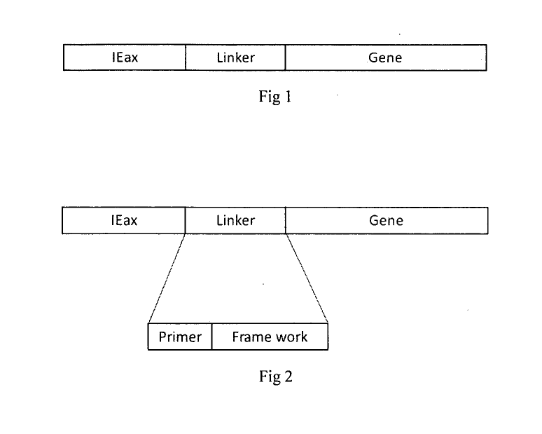

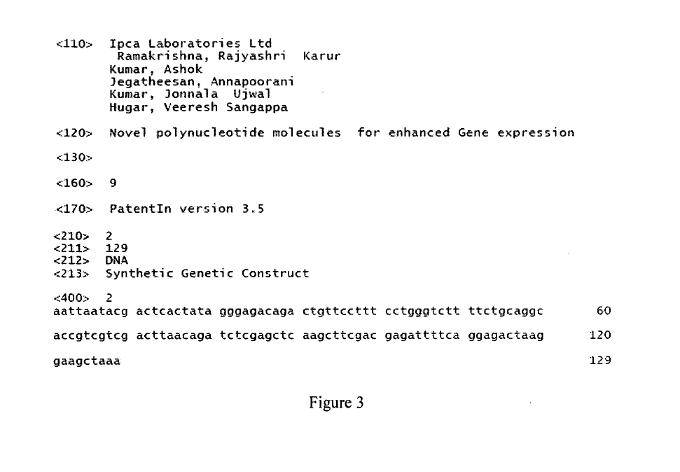

An exemplary map of polynucleotide sequence according to the invention is shown in FIG. 1. The term “operatively linked” refers to connecting one nucleic acid sequence with another nucleic acid sequence in a functional relationship. The region linking the IEax gene to nucleotide sequence encoding protein of interest may contain additional nucleotides, such as introns or enhancers for further increase of expression. An exemplary map of polynucleotide sequence with details of the linker is shown in FIG. 2. It may also contains primer attached to the promoters and frame work region requires for coding of the subject protein. According to the preferred embodiment the present invention, the novel polynucleotide molecule comprises IEax region with IPNS operatively linked to Darbepoetin gene (SEQ ID 8).

A preferred polynucleotide sequence (DNA construct) for erythropoietin is given in sequence ID 7. Another exemplary DNA construct for coding darbepoetin is represented in sequence ID No. 6. The polynucleotide sequence (molecule of the present invention) typically includes a nucleic acid sequence to be transcribed (e.g., a nucleic acid encoding a desired polypeptide) preferably with a leader sequence. Additional factors necessary or helpful in effecting expression or insertion in an expression vector may also be used as described herein. For example, the vector may also include nucleotide sequences that encode a selection marker, control sequences. Transcription termination signals, enhancers, and other nucleic acid sequences that influence gene expression, can also be included in the vector. Sequences having slight or inconsequential sequence variations from the sequences disclosed in SEQ ID 6 and SEQ ID 7 may be used which function in substantially the same manner to produce substantially the same polypeptides (protein) as the actual sequences. Erythropoietin in biological system first translated to a 166 amino acid containing polypeptide chain with arginine at position 166. In a postranslational modification, arginine 166 is cleaved by a carboxypeptidase to leave 165 amino acids. Therefore optionally polynucleotide molecule could have sequence wherein last amino acid codon is deleted. Due to code degeneracy, for example, there may be considerable variation in nucleotide sequences encoding the same amino acid sequence. The variations may be attributable to local mutations or structural modifications. Methods for introducing mutations into nucleotide sequences are well known to those skilled in the art (Molecular cloning: a laboratory manual., 3rd ed., Joseph Sambrook, David W. Russell., Cold Spring Harbor Laboratory Press, 2001).

The polynucleotide sequence of the invention or its functional oligo-nucleotide fragment thereof may be hydrogen bonded to a complementary nucleotide base sequence, and an RNA made by transcription of this double stranded nucleotide sequence, are part of the present invention.

The polynucleotide sequence of the invention may by constructed by chemical synthesis or enzymatic ligation reactions carried out by procedures known in the art. The polynucleotide sequence may include a number of restriction enzyme recognition sites or linker sequences. Nucleic acid molecules encoding the subject protein may be separately synthesized either chemically or may be cloned from a genomic or cDNA mammalian library using oligonucleotide probes derived from the known sequences following standard procedures. In this manner, nucleic acid molecules encoding erythropoietin may be obtained from the cells of a selected mammal and mutated to obtain structural changes. Similarly IEax Gene may also be synthesized chemically or isolated from the genome of human cytomegalovirus of native origin. Accordingly polynucleotide of invention can be synthesized chemically operatively linking any promoter upstream to IEax. Alternatively full vector can be also be synthesized to generate a vector system intergrated with the polynucleotide of the present invention. One exemplary process of preparing the desired Vector involves isolating the various elements of the vector from their biological sources or from other sources and combining them or inserting them into another vector that provides the backbone for the plasmid. The techniques used for these constructions are derived from those reported for vector construction in the prior art [Sambrook J et al.” Molecular Cloning—A laboratory Manual, Cold Spring Harbor Laboratory Press, New York, (1989)] with suitable modifications as may be necessary.

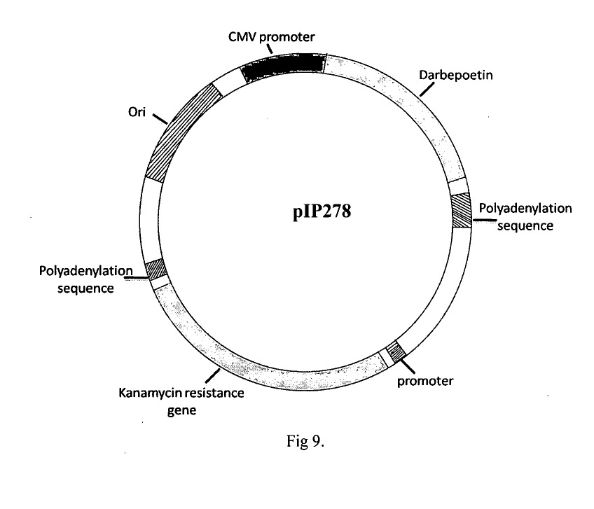

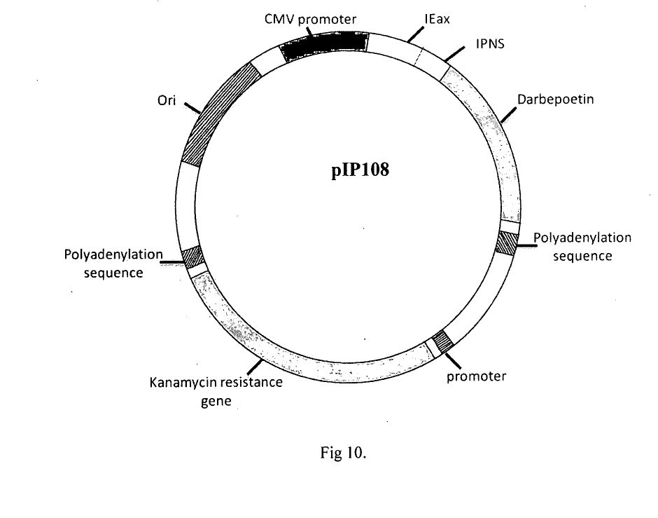

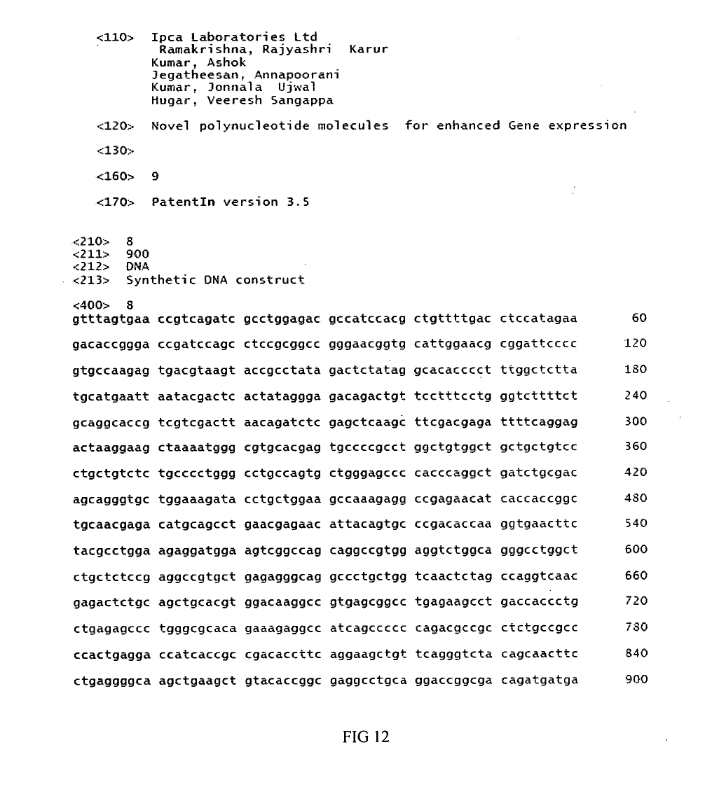

The polynucleotide sequence of the present invention is then introduced into an expression vector for transfection in mammalian cells. According to this aspect, the present invention provides expression vectors (FIGS. 9, 10) for transfection in host cells comprising the recombinant polynucleotide molecule of the present invention (FIG. 12), wherein the polynucleotide molecule comprises an IEax sequence operatively linked to the nucleotide of erythropoietin or its structural variants and regulates the level of expression of resulting protein in the host cell.

A “vector” may be any agent that is able to deliver or maintain a nucleic acid in a host cell and includes, for example, plasmids, naked nucleic acids, viral vectors, viruses, nucleic acids complexed with one or more polypeptide or other molecules, as well as nucleic acids immobilized onto solid phase particles. Vectors are described in detail below. A vector can be useful as an agent for delivering or maintaining an exogenous gene and/or protein in a host cell. A vector may be capable of transducing, transfecting, or transforming a cell, thereby causing the cell to replicate or express nucleic acids and/or proteins other than those native to the cell. A vector may include materials to aid in achieving entry of a nucleic acid into the cell, such as a viral particle, liposome, protein coating, or the like. Any method of transferring a nucleic acid into the cell may be used; unless otherwise indicated.

An “expression vector” or “Vector” typically refers to a nucleic acid construct or sequence, generated recombinantly or synthetically, with a series of specific nucleic acid elements that permit transcription of a particular nucleic acid in a host cell. The term “expression” includes any step involved in the production of the polypeptide including, but not limited to, transcription, post-transcriptional modification, translation, post-translational modification, and/or secretion. A “signal peptide” is a peptide sequence that typically precedes a polypeptide of interest and is translated in conjunction with the polypeptide and directs or facilitates the polypeptide to the secretory system. A signal peptide is typically cleaved from the polypeptide of interest following translation into protein.

According to present invention, the novel polynucleotide molecule is operatively attached to a functional promoter to get the functional assembly for expression. Vector contains promoters such as CMV, SV-40, RSV or like can be used and well known in the art. In one of the embodiment of the invention polynucleotide molecule is attached with a vector containing CMV promoter, and thus the expression vector is made by operatively linking Sequence ID 8 or 9 to the CMV promoter (as represented in FIG. 10).

The transfer/insertion of the polynucleotide molecule of the present invention to the respective vector may be accomplished by the process commonly explained as the restriction and ligation reactions with the help of the restriction and ligase enzymes. These enzymes are widely used in the recombinant DNA technology for creating a gap and joining of two competent gene segments. Restriction enzymes utilization is based on the sequence of DNA utilized and manipulative requirement of the segment of the DNA. Further it should be appreciated that DNA may be modified based on the specific requirement and restriction pattern with optional multiple cloning site. Exemplary restriction enzyme used in the present invention include Hind III and Not I.

Further the expression vector includes the amplification/selection marker gene, the selection of marker gene may depends on the host organisms viz. prokaryotic or eukaryotic host system. In present invention marker gene was selected from the group comprising of, but not limited to, drug resistant markers like kanamycin, ampicillin, neomycin, and the like. In addition, the expression vector comprises the origin of replication, poly adenylation sequence and other functional components of general vector system.

Copies of the expression vector may be produced by transforming it in a prokaryotic host cell, more preferably in E. coli. Transfer vector may be isolated from E. coli by methods known in the art for example electroporation, chemical mediated, lypofection, bombardment etc. In one of the embodiment of the invention E. coli has been utilized for the same purpose with electroporation and chemical competency.

In a further aspect, the present invention also provides stable host cell systems transfected with the expression vector comprising the recombinant DNA molecule (polynucleotide molecule of the present invention) having the Expression regulatory IEax sequence region operatively linked to the nucleotide encoding erythropoietin or its structural variants, including Darbepoetin.

The recombinant polynucleotide molecule of the invention may be transfected in a wide variety of mammalian cells expressing the erythropoietin and its structural variants. The Host cells may include eukaryotic cells which are selected form the group comprises of but not limited to, CHO cells, CHO-S cells, BHK, NS0, PER.C6, HEK 293, among others. It may be preferred to use CHO or CHO-S cells due to its wide acceptability and easy availability. The transfection of the expression vector of the present invention to the host cell may be carried by methods generally known in the technology. These include electroporation, gene gun method, chemical mediated transfer, etc. For production of active therapeutic protein gene should be placed inside the eukaryotic host cell. Integration site of the vector have sometimes importance in accordance to get good expression, which is the further useful aspect of the invention. In one embodiments of the present invention CHO, CHO-S cells are transfected with the Vector by the process of lipofectamin LTX reagent, and Vector DNA with mixing thoroughly according to requirement of the reaction.

Culturing media for CHO cells generally comprises, serum containing media and serum free media. Within the serum containing media Fetal bovine serum (FBS) and bovine serum albumin (BSA) are probably the most extensively utilized serum for mammalian cell culture, although other mammalian sera are used. Serum is known to contain many major components including albumin and transferrin and also minor components many of which have not been fully identified nor their action determined, thus serum will differ from batch to batch possibly requiring testing to determine levels of the various components and their effect on the cells. In serum free media components of the medium are mostly inorganic, synthetic or recombinant and as such are not obtained directly from any animal source.

Culturing condition for CHO or CHO-S cells generally comprises providing sterilized media according to the cell requirement. CHO cells can be cultivated in the adherent or suspension culture, for suspension culture T flasks are generally incubated at 36.5° C. in 5% CO₂ incubator.

Under standard conditions of culturing the host cell containing the polynucleotide molecule of the present invention expresses the protein of interest in higher concentration levels. Selection of cell lines having product gene depends on various parameters including antibiotic resistance from vector, deficient cell line, fluorescence, color change, production detection etc. In one of the embodiment cells are selected by the utilization of G148 containing media. Furthermore after selecting few numbers of colonies the culture plates are evaluated for quantification of product produced biologically, using any suitable technique for instance ELISA test. Double antibody sandwich ELISA method, was utilized to select the best expressing clones. The cells to be tested for expression may be seeded such that they reach 70-80% confluence the next day. The test may be preformed according to the standard protocol utilizing mouse monoclonal antibody to human EPO, rabbit polyclonal antibody to human EPO and goat anti-rabbit IgG alkaline phosphatase. Eventually product can be characterized by various possible means including SDS-PAGE, Western Blot.

Cells producing high levels of protein may be identified and selected and subcloned to establish long term cell lines from the selected cells and; the selected cells may be cultured in a suitable medium until sufficient amounts of erythropoietin are produced by the cell. The cells transfected with the expression vector of the present invention are capable of expressing recombinant protein in vitro at levels of at least 500 IU/ml/24 hours, preferably at least 3200 IU/ml/24 hrs and most preferably about 32000-96000 IU/ml/72 hrs, and it is further expected to give enhanced level of expression through selection and methods well known in skill and art.

Given the teaching of the present invention, it is possible to transfect mammalian host cells with the novel DNA construct of the present invention, whereby the cell become capable of expressing the subject proteins at higher concentration levels. The transfected construct gets integrated into the host cell gene pool. By virtue of the property of the cells, when it divides or produces daughter cells, the cells are capable of transferring the DNA of the present invention to its daughter cell, thus making a reproducible new clone expressing protein at very high levels. Accordingly, the invention also encompasses to new mammalian cells stably transfected with an expression vector of the invention. The term “stably transfected” refers to stable integration of the recombinant polynucleotide molecule of the invention into the genome of the host cell.

The present invention further include the use of the high expressing clone of mammalian cells containing the polynucleotide molecule of the present invention for the production of erythropoietin or its structural variants. The method to produce high levels of erythropoietin or its structural variants comprises culturing the high expressing host cells strain of the present invention in a suitable harvesting medium until sufficient amounts of erythropoietin are produced in the medium and separating the erythropoietin or structural variants obtained.

Accordingly to a preferred embodiment, a method to produce high expression of Darbepoetin is provided comprising the following:
1. culturing a host cell transfected with an expression vector containing expression regulatory polynucleotide lEax sequence operatively linked to nucleotide sequence encoding Darbepoetin (sequence ID No.5) in a suitable medium for sufficient period.
2. Separating Darbepoetin from the cell and culturing medium; and
3. Purifying Darbepoetin.

Accordingly in another preferred embodiment, a method to produce high expression of Erythropoetin is provided comprising the following:
1. culturing a host cell transfected with an expression vector containing expression regulatory polynucleotide IEax sequence operatively linked to nucleotide sequence encoding Erythropoetin (sequence ID No.4) in a suitable medium for sufficient period;
2. Separating Erythropoetin from said cell and culturing medium; and
3. Purifying Erythropoetin.

Cells producing high levels of Darbepoetin may be identified and selected and subcloned to establish long term cell lines from the selected cells and an expression level of at least 500 IU/ml/24 hours, preferably at least 3200 IU/ml/24 hrs and most preferably about 32000-96000 IU/ml/72 hrs or more can be achieved. Biologically active Darbepoetin expressed may be assayed by known procedures such as ELISA. Erythropoietin or darbepoetin produced are isolated and purifiedthrough the procedures well known in the present art, the process generally includes ultrafiltration, flat-bed electrofocusing, gel filtration, electrophoresis, isotachophoresis, general/cation/ion exchange/size-exclusion chromatography, affinity chromatography, HPLC methods, lyophilization methods, centrifugation methods etc. See for example, Introduction to modern liquid chromotography, 3^{rd} edition, Lloyd R. Snyder (et. al.) Wiley & Sons, Inc. 2009 (ISBN 9780470167540); Scopes, Protein Purification: Principles and Practice (1994); Molecular cloning: a laboratory manual., 3rd ed., Joseph Sambrook, David W. Russell., Cold Spring Harbor Laboratory Press, 2001; or Current Protocols in Molecular Biology, Ausubel, F. M., et al. (eds), John Wiley & Sons, Inc., New York. High-Performance Gradient Elution L. R. Snyder and J. W. Dolan 2007 Wiley.

Isolation can be performed for separating isoform of the glycoprotein and host cell protein and other residual protein arising through media components or cell rupture. Isoform could be isolated by utilization of the different chromatography procedures well known to skill artisan. For example reverse phase chromatography column are octadecyl carbon chain (C18) bonded silica, C8 bonded silica, C4 bonded silica, pure silica (L3-88 columns), are useful to remove host cell proteins. Typical anion exchanger resins, which can be employed to purify glycoprotein isoforms, comprise functional groups like diethylaminoethyl (DEAE), which are for example: DEAE SEPHAROSE (Amersham Biosciences); quaternary aminoethyl; quaternary ammonium, for example: Q SEPHAROSE XL (AmershamBiosciences); dimethylaminoethyl (DMAE), trimethylaminoethyl (TMAE) for example ‘FRACTOGEL® EMD DEAE (Merck) etc.

Mobile phases can be selected form various buffers, solvents having polarity concentrations. Some of them are phosphate buffers, acetate, sulphate, halide, aqueous non-aqueous organic salt (s) or solvent (s), aqueous non-aqueous inorganic salt (s) or solvent (s). Generally mobile phases are buffer systems which maintain certain parameters in the column to separate molecule efficiently. For example ions of choice might be various possible NaCl H₃PO₄, fe+ carbonates, cu+ sulphates etc. further pH concentration plays an important role for the separation of the protein which can be selected from the range of 3.0-8.0. Skill artisan has to perform various tests to select suitable mobile phase (s), salt ion system (s), pH range to effectively isolate desired protein.

In yet another aspect of the invention utilization of the culture conditions and components of the media are experimented. The media optimization for the best possible utilization of the production capability of the utilized host cell has been tested. In one of the embodiment of the invention eukaryotic host cell is exemplified by CHO cell. CHO cell line obtained from commercial sources or culture banks can be maintained vital in culture conditions, the process used to culture the cell can be divided into two approaches namely serum free media (no plasma) and serum contained media (having plasma). The serum free media generally includes chemical factors and components to generated environment and nutritional pool for CHO cell. The exemplified media is generally utilized in the art to culture the CHO cell in the artificial system, or bioreactor. This invention also encompasses the all possible media compositions where the expression of the protein is concerned when one of the cells transfected with the expression vector.

In another aspect of the invention CHO/CHO-S cells can be comprising of functional enzyme or its gene such as β-Galactoside-α-2,6-sialyltrransferase, giving specific sialic acid α-2,6-linkage or glycosylation leading to various isoforms of said protein with said polynucleotide sequence. Compounds and enzyme which are not part of the host cell may be supplied into the culture medium to form required glycosylation and may provide advantage to obtain required isoform. In particular N-glycosylation or O-glycosylation activators activators/inhibitors may be incorporated to tailor the N- or O-glycosylation pattern in the resulting protein.

In yet another aspect of the invention product glycosylation pattern was modulated through various techniques well known in the art, for example, incorporation of Manganese in the media, use of sialidase inhibitor, addition of chemical compound or factors in culture media such as ammonium chloride etc. Furthermore conditions management wherein specific isoform of the product is recovered in larger concentrations is utilized.

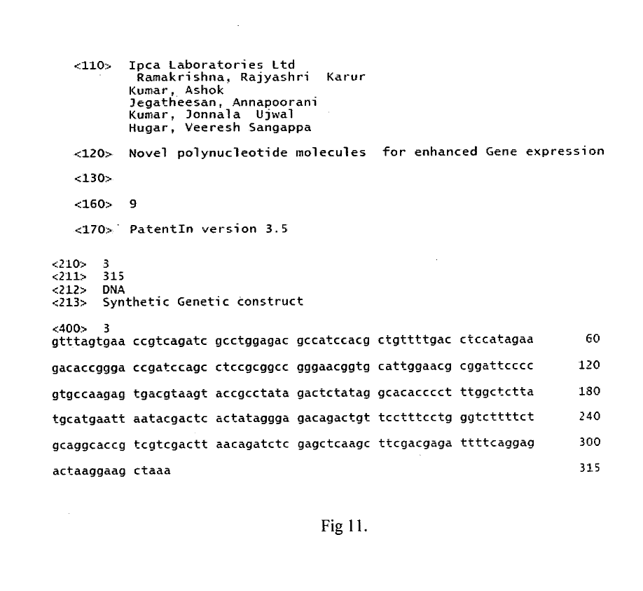

For comparison purpose, the present invention used general vector consisting CMV promoter which is known for production of darbepoetin. Expression vector was made by insertion of an optimized gene of the darbepoetin (sequence ID No. 6) in present vector by standard manner hereinafter referred as pIP278. Expression vector (pIP108) containing the polynucleotide molecule of the present invention (sequence ID No. 8) with CMV promoter is made as described hereinbefore. Both vectors were transfected in to CHO cell and the expression levels were studied. The results are represented in table 1 given below, and gel picture of the expression levels are displayed in FIG. 11.

TABLE 1

| | | With SEQ ID No. 6 | With SEQ ID No. 8 |
|---|---|---|---|
| | | in expression | in expression |
| | | vector (IU/ | vector (IU/ |
| Sl. No. | Cell line | ml/24 hours) pIP278-DB | ml/24 hours) pIP108-DB |
| 1 | CHO | 60 | 520 |
| 2 | CHO | 69 | 592 |
| 3 | CHO | 70 | 890 |
| 4 | CHO | 75 | 997 |
| 5 | CHO | 115 | 1770 |
| 6 | CHO | 120 | 3200 |

Recombinant protein produced biologically, differs from one another if the production process is slightly modified in any aspect. In biological system when proteins are produced, they are folded and modified within the cytoplasm commonly referred as “post-translational modification”. Due to difference in the biological cell line, media composition, production process condition the difference in the post translational condition may arise which eventually may give rise to unique, folding of protein, glycosylation, groups attached to protein which may further enhance the activity of the final protein or vice-versa. Recombinant protein produced by the utilization of polynuceotide molecules, expression vector, and host cells of the present invention encompasses the same biosimilars product. As exemplified in one of the embodiment the erythropoietin or its isoform produced is also a biosimilar and different from the commonly available erythropoietin or its isoform.

Also comprehended by the invention are pharmaceutical compositions comprising a therapeutically effective amount of a product produced according to the present invention. The compositions may also include suitable diluents, adjuvant and/or carrier useful in therapy. Pharmaceutical compositions comprising a therapeutically effective amount of an erythropoietin analog together with a suitable diluent, adjuvant and/or carrier are also encompassed. A “therapeutically effective amount” as used herein refers to that amount which provides therapeutic effect for a given condition and administration regimen. The administration of isoforms of human erythropoietin or erythropoietin analogs is preferably by parenteral routes. The specific route chosen will depend upon the condition being treated. The administration of isoforms of human erythropoietin or erythropoietin analogs is preferably done as part of a formulation containing a suitable carrier, such as human serum albumin, a suitable diluent, such as a buffered saline solution, and/or a suitable adjuvant. The required dosage will be in amounts sufficient to raise the hematocrit of subjects and will vary depending upon the severity of the condition being treated, the method of administration used and the like.

In another embodiment, the invention provides use of polynucleotide molecule of the present invention encoding human erythropoietin or recombinant Darbepoetin in the mammalian cell system, in a medicament for curative action in subject suffering from anemia with chronic renal failure.

The recombinant polynucleotide molecules, expression vectors and transformed mammalian cells of the invention may also have useful applications in gene therapy, whereby a functional gene of the protein for example, darbepoetin is introduced into a mammal in need thereof, for example mammals having anemias. Thus, the recombinant polynucleotide molecule of the invention may be used in gene therapy as briefly described below. The recombinant polynucleotide molecule may be introduced into cells of a mammal, for example Haemopoietic stem cells removed from the bone marrow or blood of the mammal. Haemopoietic stem cells are particularly suited to somatic gene therapy as regenerative bone marrow cells may be readily isolated, modified by gene transfer and transplanted into an immuno compromised host to reconstitute the host's Haemopoietic system.

Following examples are offered to more fully illustrate the invention, but are not to be construed as limiting the scope thereof.

### Example 1

### Polynucleotide of Darbepoetin Gene (DB) Vector Creation

The polynucleotide sequence (Sequence ID 8) operatively linked with CMV promoter was synthesized chemically with oligosynthesizer.

The synthetic sequence was cloned into the vector consisting of control sequences, origin of replication, polyadenylation sequences, additional restriction sites, selection marker gene kanamycin and accessory elements to generate the construct pIP108-DB. The ligation mix was transformed into chemically competent DH5-α cells and plated on LB-Kanamycin plates. The plates were incubated at 37° C. overnight.

Plasmid was prepared from the clones obtained and the sequence of the insert confirmed by sequencing. Thus, the construct pIP108-DB was made.

### Darbepoetin Gene (DB) in Conventional Vector

The darbepoetin gene synthesized chemically (SEQ ID 6) along with CMV promoter, as disclosed earlier was inserted into the conventional vector consisting of control sequences, origin of replication, polyadenylation sequences, additional restriction sites, selection marker gene kanamycin and accessory elements to generate Vector pIP278-DB. The ligation mix was transformed into chemically competent DH5-α cells and plated on LB-ampicillin plates. Large scale DNA preparation was made from the clones obtained and full length sequencing of the constructs performed to confirm the sequence of the clone/s obtained.

### Example 2

### Generation of stable Cell Lines

CHO cells were cultured in RPMI complete medium till the cells reached 60-80% confluence. 2 ml of the RPMI complete medium containing 0.5 million cells/ml were plated onto 6-well plate on the day before the transfection. −2.0 μg of DNA to be transfected was diluted with 500 μl of the Opti-MEM. 6.25 μl of Lipofectamin LTX reagent was added and the mixture was incubated at room temperature for 30 min. The mixture was added to the plated cells in serum free, antibiotic free medium. The cells were incubated at 37° C. in 5% CO2 Incubator for 6-8 hrs. After this period of incubation, the cells were supplemented with complete medium and the cells cultured for further 12 hrs. The cells were selected on different concentrations of geneticin for a period of 16 days with subculturing done every 2 days till complete cell death was observed in the non-transfected controls. The clones were subjected to ELISA for determining the concentration of EPO/isomers produced by the cell line.

The mixed cell line showing maximum expression of EPO or its isomer was subjected to limiting dilution. Pure clones were isolated and the expression of EPO/isomer evaluated. A number of copies of the highest expressing cell lines were frozen for further use.

### Determination of Transient Expression in CHO Cells

The transfected CHO cells were fixed in 3.5% formaldehyde in phosphate buffered saline, pH 7.4 for 10 mins at room temperature and washed thrice in PBS buffer. The washed cells were incubated with 1% BSA in PBS overnight at 4° C. The cells were washed thrice with PBS and incubated with Rabbit polyclonal anti-human EPO antibody (diluted 1:20) in PBS for 2 hrs at room temperature. Antibodies were washed in PBS thrice, and the cells further incubated with goat-anti-rabbit-IgG-FITC conjugates for 45 minutes at room temperature. The cells were, washed thrice in PBS and observed under fluorescent microscope.

### Detection and Quantitation of EPO/its Isomers

EPO and its isomers. Appropriate volume of the supernatant was used for quantitating the amount of EPO/darbepoetin produced using the R&D System Quantikine kit. The protocol as recommended by the manufacturers was used without modifications. Dilutions were made as required using the specimen dilution buffer in the kit. The amount of EPO/isomer produced was quantitated against the standards provided in the kit.

### SDS-PAGE

10% acrylamide gel in Tris-glycine buffer was prepared according to Sambrook et. al. (1996). Required amount of proteininprotein gel loading dye was boiled for 5 min and loaded on the gel. Mini gels were electrophoresed at appropriate voltage and the gels were either used for western transfer or were stained with coomasie blue or silver stained following standard protocols (Sambrook et. al. 1996).

### Western Transfer

The SDS-PAGE gel to be transferred was electrophoresed as described above. The gel was fixed in Towbin buffer and semi-dry transfer of the gel on to PVDF membrane was done as recommended by BioRad. The membrane was dried and used for hybridization to appropriate antibodies.

### Western Hybridization

Western hybridization protocol, as recommended by Abcam, was followed. The Western blot was blocked with TBST containing 5% non-fat milk overnight at 4° C. Fresh TBST containing 1:500 dilution of the required antibody was added to the membrane and the membrane rocked at room temperature (RT) for 2-4 hrs. The blot was washed at RT for 10 mins thrice in TBST and then bound to 1:1000 dilution of secondary antibody conjugate for 2 hrs at RT with rocking. The blot was washed again for 10 minutes thrice in TBST at RT, and 2 ml of NBT/BCIP added to it. The antigen-antibody complex was detected as a blue colored band.

### Example 3

### Production and Isolation of Darbepoetin

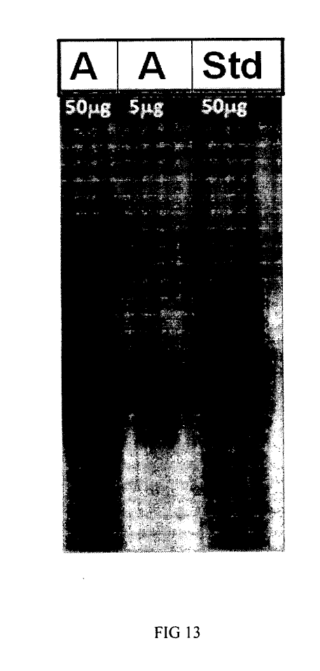

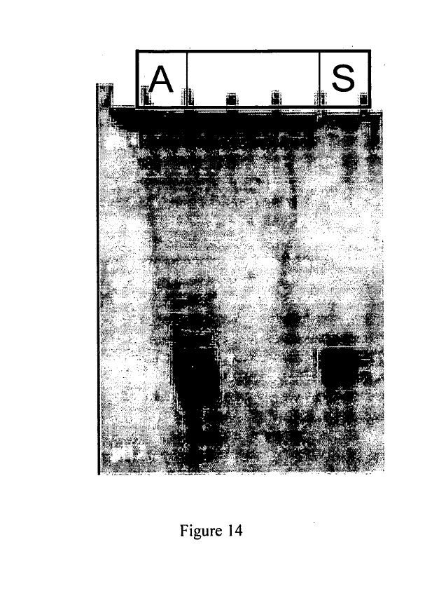

Host production cell lines (CHO) are selected from transient expressing pool. Research cell bank was prepared for continual supply of the cell line for experiment. cryo-vial of host cell line was revived for seed preparation for lab scale production of product. The cells expressing erythropoietin isoform are cultured from the research cell bank in 3 step process of T flasks before being inoculated into the lab scale reactor. Thawed cell line was inoculated at initial cell count of about 0.5 million to 5 ml of the CD medium (invitrogen Cat. No. 12681) in a T25 and allowed to grow at 37° C. for 2 days in CO₂ incubator. Cells are allowed to grow till they reach about 4 million cells. Further similar procedure was followed for T75 and T150 flask with same cell count inoculation and allowed to grow in similar parameter conditions till they reach about 4 million cells concentration. After 3 stages of T flask transfer seed is pooled and transferred with cell count of about 0.2 million to lab scale bioreactor. Similar media was taken for bioreactor experiment having initial pH of about 7. Temperature of culture is controlled at about 37.0° C. Dissolved oxygen levels were maintained at constant levels and feeding glucose level was adjusted to high levels in growth phase and reduced during production phase to maintain good cell viability and production. Culture was harvested on day 10 to collect the broth containing desired product. Harvested broth immediately cooled and cells were centrifuged at about 5000 rpm for about 5 minutes under 4° C. Cell pellet generated is discarded and supernatant is filtered through a 1.2μ A filter, subsequently with 0.2μ filter under sterile conditions. Filtrate is further subjected to ion exchange chromatography to isolate & purify darbepoetin. The product character match with Darbepoetin (from Amgen) based on the SDS page and IEF comparison as shown in FIG. 13 and FIG. 14.

### DEPOSIT INFORMATION

E. coli culture transfected with Vector pIP108 consisting of the polynucleotide of the present invention was deposited with the Microbial Type Culture Collection and Gene Bank (MTCC) on June 2 of 2009 having deposit number MTCC 5478 at Institute of Microbial Technology (IMTECH) Council of Scientific and Industrial Research (CSIR) Sector-39A Chandigarh—160 036 India, under the provision of Budapest Treaty for the international recognition of the deposit of microorganisms for the purpose of patent procedure.

CMV major immediate early Exon A and proximal
Intron A
SEQ ID 1
GTTTAGTGAACCGTCAGATCGCCTGGAGACGCCATCCACGCTGTTTTGA
CCTCCATAGAAGACACCGGGACCGATCCAGCCTCCGCGGCCGGGAACGG
TGCATTGGAACGCGGATTCCCCGTGCCAAGAGTGACGTAAGTACCGCCT
ATAGACTCTATAGGCACACCCCTTTGGCTCTTATGCATG.
IPNS
SEQ ID 2
AATTAATACGACTCACTATAGGGAGACAGACTGTTCCTTTCCTGGGTCT
TTTCTGCAGGCACCGTCGTCGACTTAACAGATCTCGAGCTCAAGCTTCG
ACGAGATTTTCAGGAGACTAAGGAAGCTAAA.
Expression Regulating Polynucleotide Sequence
with IPNS
SEQ ID 3
GTTTAGTGAACCGTCAGATCGCCTGGAGACGCCATCCACGCTGTTTTGA
CCTCCATAGAAGACACCGGGACCGATCCAGCCTCCGCGGCCGGGAACGG
TGCATTGGAACGCGGATTCCCCGTGCCAAGAGTGACGTAAGTACCGCCT
ATAGACTCTATAGGCACACCCCTTTGGCTCTTATGCATGAATTAATACG
ACTCACTATAGGGAGACAGACTGTTCCTTTCCTGGGTCTTTTCTGCAGG
CACCGTCGTCGACTTAACAGATCTCGAGCTCAAGCTTCGACGAGATTTT
CAGGAGACTAAGGAAGCTAAA
Erythropoietin
SEQ ID 4
APPRLICDSRVLERYLLEAKEAENITTGCAEHCSLNENITVPDTKVNFY
AWKRMEVGQQAVEVWQGLALLSEAVLRGQALLVNSSQPWEPLQLHVDKA
VSGLRSLTTLLRALGAQKEAISPPDAASAAPLRTITADTFRKLFRVYSN
FLRGKLKLYTGEACRTGDR
Darbepoetin
SEQ ID 5
APPRLICDSRVLERYLLEAKEAENITTGCNETCSLNENITVPDTKVNFY
AWKRMEVGQQAVEVWQGLALLSEAVLRGQALLVNSSQVNETLQLHVDKA
VSGLRSLTTLLRALGAQKEAISPPDAASAAPLRTITADTFRKLFRVYSN
FLRGKLKLYTGEACRTGDR
Optimized Darbepoetin DNA sequence
SEQ ID 6
ATGGGCGTGCACGAGTGCCCCGCCTGGCTGTGGCTGCTGCTGTCCCTGC
TGTCTCTGCCCCTGGGCCTGCCAGTGCTGGGAGCCCCACCCAGGCTGAT
CTGCGACAGCAGGGTGCTGGAAAGATACCTGCTGGAAGCCAAAGAGGCC
GAGAACATCACCACCGGCTGCAACGAGACATGCAGCCTGAACGAGAACA
TTACAGTGCCCGACACCAAGGTGAACTTCTACGCCTGGAAGAGGATGGA
AGTCGGCCAGCAGGCCGTGGAGGTCTGGCAGGGCCTGGCTCTGCTCTCC
GAGGCCGTGCTGAGAGGGCAGGCCCTGCTGGTCAACTCTAGCCAGGTCA
ACGAGACTCTGCAGCTGCACGTGGACAAGGCCGTGAGCGGCCTGAGAAG
CCTGACCACCCTGCTGAGAGCCCTGGGCGCACAGAAAGAGGCCATCAGC
CCCCCAGACGCCGCCTCTGCCGCCCCACTGAGGACCATCACCGCCGACA
CCTTCAGGAAGCTGTTCAGGGTCTACAGCAACTTCCTGAGGGGCAAGCT
GAAGCTGTACACCGGCGAGGCCTGCAGGACCGGCGACAGATGATGAGCG
GCCGC
Optimized Erythropoietin DNA sequence
SEQ ID 7
ATGGGCGTGCACGAGTGCCCCGCCTGGCTGTGGCTGCTGCTGTCCCTGC
TGTCTCTGCCCCTGGGCCTGCCAGTGCTGGGAGCCCCACCCAGGCTGAT
CTGCGACAGCAGGGTGCTGGAAAGATACCTGCTGGAAGCCAAAGAGGCC
GAGAACATCACCACCGGCTGCGCCGAGCACTGCAGCCTGAACGAGAACA
TTACAGTGCCCGACACCAAGGTGAACTTCTACGCCTGGAAGAGGATGGA
AGTCGGCCAGCAGGCCGTGGAGGTCTGGCAGGGCCTGGCTCTGCTCTCC
GAGGCCGTGCTGAGAGGGCAGGCCCTGCTGGTCAACTCTAGCCAGCCCT
GGGAGCCCCTGCAGCTGCACGTGGACAAGGCCGTGAGCGGCCTGAGAAG
CCTGACCACCCTGCTGAGAGCCCTGGGCGCACAGAAAGAGGCCATCAGC
CCCCCAGACGCCGCCTCTGCCGCCCCACTGAGGACCATCACCGCCGACA
CCTTCAGGAAGCTGTTCAGGGTCTACAGCAACTTCCTGAGGGGCAAGCT
GAAGCTGTACACCGGCGAGGCCTGCAGGACCGGCGACAGATGATGAGCG
GCCGCGACTCTAGCGGCCGC
Optimized Darbepoetin polynucleotide molecule
SEQ ID 8
GTTTAGTGAACCGTCAGATCGCCTGGAGACGCCATCCACGCTGTTTTGA
CCTCCATAGAAGACACCGGGACCGATCCAGCCTCCGCGGCCGGGAACGG
TGCATTGGAACGCGGATTCCCCGTGCCAAGAGTGACGTAAGTACCGCCT
ATAGACTCTATAGGCACACCCCTTTGGCTCTTATGCATGAATTAATACG
ACTCACTATAGGGAGACAGACTGTTCCTTTCCTGGGTCTTTTCTGCAGG
CACCGTCGTCGACTTAACAGATCTCGAGCTCAAGCTTCGACGAGATTTT
CAGGAGACTAAGGAAGCTAAAATGGGCGTGCACGAGTGCCCCGCCTGGC
TGTGGCTGCTGCTGTCCCTGCTGTCTCTGCCCCTGGGCCTGCCAGTGCT
GGGAGCCCCACCCAGGCTGATCTGCGACAGCAGGGTGCTGGAAAGATAC
CTGCTGGAAGCCAAAGAGGCCGAGAACATCACCACCGGCTGCAACGAGA
CATGCAGCCTGAACGAGAACATTACAGTGCCCGACACCAAGGTGAACTT
CTACGCCTGGAAGAGGATGGAAGTCGGCCAGCAGGCCGTGGAGGTCTGG
CAGGGCCTGGCTCTGCTCTCCGAGGCCGTGCTGAGAGGGCAGGCCCTGC
TGGTCAACTCTAGCCAGGTCAACGAGACTCTGCAGCTGCACGTGGACAA
GGCCGTGAGCGGCCTGAGAAGCCTGACCACCCTGCTGAGAGCCCTGGGC
GCACAGAAAGAGGCCATCAGCCCCCCAGACGCCGCCTCTGCCGCCCCAC
TGAGGACCATCACCGCCGACACCTTCAGGAAGCTGTTCAGGGTCTACAG
CAACTTCCTGAGGGGCAAGCTGAAGCTGTACACCGGCGAGGCCTGCAGG
ACCGGCGACAGATGATGA
Optimized Erythropoietin polynucleotide molecule
SEQ ID 9
GTTTAGTGAACCGTCAGATCGCCTGGAGACGCCATCCACGCTGTTTTGA
CCTCCATAGAAGACACCGGGACCGATCCAGCCTCCGCGGCCGGGAACGG
TGCATTGGAACGCGGATTCCCCGTGCCAAGAGTGACGTAAGTACCGCCT
ATAGACTCTATAGGCACACCCCTTTGGCTCTTATGCATGAATTAATACG
ACTCACTATAGGGAGACAGACTGTTCCTTTCCTGGGTCTTTTCTGCAGG
CACCGTCGTCGACTTAACAGATCTCGAGCTCAAGCTTCGACGAGATTTT
CAGGAGACTAAGGAAGCTAAAATGGGCGTGCACGAGTGCCCCGCCTGGC
TGTGGCTGCTGCTGTCCCTGCTGTCTCTGCCCCTGGGCCTGCCAGTGCT
GGGAGCCCCACCCAGGCTGATCTGCGACAGCAGGGTGCTGGAAAGATAC
CTGCTGGAAGCCAAAGAGGCCGAGAACATCACCACCGGCTGCGCCGAGC
ACTGCAGCCTGAACGAGAACATTACAGTGCCCGACACCAAGGTGAACTT
CTACGCCTGGAAGAGGATGGAAGTCGGCCAGCAGGCCGTGGAGGTCTGG
ACAGGGCCTGGCTCTGCTCTCCGAGGCCGTGCTGAGAGGGCGGCCCTGC
TGGTCAACTCTAGCCAGCCCTGGGAGCCCCTGCAGCTGCACGTGGACAA
GGCCGTGAGCGGCCTGAGAAGCCTGACCACCCTGCTGAGAGCCCTGGGC
GCACAGAAAGAGGCCATCAGCCCCCCAGACGCCGCCTCTGCCGCCCCAC
TGAGGACCATCACCGCCGACACCTTCAGGAAGCTGTTCAGGGTCTACAG
CAACTTCCTGAGGGGCAAGCTGAAGCTGTACACCGGCGAGGCCTGCAGG
ACCGGCGACAGATGATGAGCGGCCGCGACTCTA.

1-22. (canceled)

23. A polynucleotide comprising human cytomegalovirus Exon A and proximal Intron A of major immediate early gene, or their functional variants, operatively linked to a nucleotide sequence encoding erythropoietin, darbepoietin, or structural variants thereof.

24. The polynucleotide comprising human cytomegalovirus Exon A and proximal Intron A of major immediate early gene of their functional variants as claimed in claim 23, wherein the proximal Intron A ends at a first restriction site of SphI of Intron A characterized by sequence starting from 1265 and ending 1316 of human cytomegalovirus major immediate early gene.

25. The polynucleotide of claim 23, wherein the human cytomegalovirus Exon A and proximal Intron A of major immediate early gene are linked upstream to the nucleotide sequence encoding erythropoietin, darbepoietin, or structural variants thereof.

26. The polynucleotide of claim 25, wherein the human cytomegalovirus Exon A and proximal Intron A of major immediate early gene are linked to the nucleotide sequence encoding erythropoietin, darbepoietin, or structural variants thereof via a linker sequence.

27. The polynucleotide of claim 26, wherein the linker sequence is SEQ ID NO: 2 or functional variants thereof.

28. The polynucleotide of claim 23, comprising SEQ ID NO: 3 operatively linked to the nucleotide sequence encoding erythropoietin, darbepoietin, or structural variants thereof.

29. The polynucleotide of claim 23, wherein the human cytomegalovirus Exon A and proximal Intron A of major immediate early gene or their functional variants comprising at least about 315 bp.

30. The polynucleotide of SEQ ID NO: 6, SEQ ID NO: 7, SEQ ID NO: 8, or SEQ ID NO: 9.

31. A vector comprising a polynucleotide having a human cytomegalovirus Exon A and proximal Intron A of major immediate early gene, or their functional variants, operatively linked to a nucleotide sequence encoding erythropoietin, darbepoietin, or structural variants thereof.

32. The vector of claim 31, wherein the human cytomegalovirus Exon A and proximal Intron A of major immediate early are linked to a nucleotide sequence encoding erythropoietin, darbepoietin, or structural variants thereof via a linker sequence.

33. The polynucleotide of claim 32, wherein the linker sequence is SEQ ID NO: 2 or functional variants thereof.

34. A host cell comprising a polynucleotide containing a human cytomegalovirus Exon A and proximal Intron A of major immediate early gene, or their functional variants, operatively linked to a nucleotide sequence encoding erythropoietin, darbepoietin, or structural variants thereof.

35. The host cell of claim 34, wherein the human cytomegalovirus Exon A and proximal Intron A of major immediate early gene are linked to a nucleotide sequence encoding erythropoietin, darbepoietin, or structural variants thereof via a linker sequence.

36. The polynucleotide of claim 35, wherein the linker sequence is SEQ ID NO: 2 or functional variants thereof.

37. The host cell of claim 34, wherein the cell is a CHO cell line.

38. A process for producing erythropoetin, darbepoetin or structural variants thereof, comprising the steps of:
(a) incorporating a polynucleotide into a host cell, wherein the polynucleotide contains a human cytomegalovirus Exon A and proximal Intron A of major immediate early gene, or their functional variants, operatively linked to a nucleotide sequence encoding erythropoetin, darbepoetin or structural variants thereof; and
(b) culturing the host cell in a suitable growth medium to produce erythropoetin, darbepoetin or structural variants thereof.

39. The process of claim 38, further comprising the step of (c) separating and/or purifying the said erythropoetin, darbepoetin or structural variants thereof.

40. A composition comprising erythropoetin, darbepoetin or structural variants thereof encoded from a polynucleotide comprising human cytomegalovirus Exon A and proximal Intron A of major immediate early gene or their functional variants according to claim 23.

41. Method of increasing hematocrit levels in mammals comprising administration of erythropoietin, darbepoetin or structural variants thereof encoded from a polynucleotide according to claim 23.

42. Use of biologically active erythropoietin or darbepoetin or structural variants thereof encoded from polynucleotide according to claim 23.


SEQUENCE LISTING (9 sequences)

SEQ ID NO: 1 - DNA, 186 bp, Human cytomegalovirus
gtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatg

SEQ ID NO: 2 - DNA, 129 bp, Artificial sequence
  completely synthesized
aattaatacgactcactatagggagacagactgttcctttcctgggtcttttctgcaggcaccgtcgtcgacttaacagatctcgagctcaagcttcgacgagattttcaggagactaaggaagctaaa

SEQ ID NO: 3 - DNA, 315 bp, Artificial Sequence
  completely synthesized
gtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaattaatacgactcactatagggagacagactgttcctttcctgggtcttttctgcaggcaccgtcgtcgacttaacagatctcgagctcaagcttcgacgagattttcaggagactaaggaagctaaa

SEQ ID NO: 4 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 5 - protein, 166 aa, Artificial Sequence
  partially synthesized
APPRLICDSRVLERYLLEAKEAENITTGCNETCSLNENITVPDTKVNFYAWKRMEVGQQAVEVWQGLALLSEAVLRGQALLVNSSQVNETLQLHVDKAVSGLRSLTTLLRALGAQKEAISPPDAASAAPLRTITADTFRKLFRVYSNFLRGKLKLYTGEACRTGDR

SEQ ID NO: 6 - DNA, 593 bp, Artificial Sequence
  partially synthesized
atgggcgtgcacgagtgccccgcctggctgtggctgctgctgtccctgctgtctctgcccctgggcctgccagtgctgggagccccacccaggctgatctgcgacagcagggtgctggaaagatacctgctggaagccaaagaggccgagaacatcaccaccggctgcaacgagacatgcagcctgaacgagaacattacagtgcccgacaccaaggtgaacttctacgcctggaagaggatggaagtcggccagcaggccgtggaggtctggcagggcctggctctgctctccgaggccgtgctgagagggcaggccctgctggtcaactctagccaggtcaacgagactctgcagctgcacgtggacaaggccgtgagcggcctgagaagcctgaccaccctgctgagagccctgggcgcacagaaagaggccatcagccccccagacgccgcctctgccgccccactgaggaccatcaccgccgacaccttcaggaagctgttcagggtctacagcaacttcctgaggggcaagctgaagctgtacaccggcgaggcctgcaggaccggcgacagatgatgagcggccgc

SEQ ID NO: 7 - DNA, 608 bp, Artificial Sequence
  partially synthesized
atgggcgtgcacgagtgccccgcctggctgtggctgctgctgtccctgctgtctctgcccctgggcctgccagtgctgggagccccacccaggctgatctgcgacagcagggtgctggaaagatacctgctggaagccaaagaggccgagaacatcaccaccggctgcgccgagcactgcagcctgaacgagaacattacagtgcccgacaccaaggtgaacttctacgcctggaagaggatggaagtcggccagcaggccgtggaggtctggcagggcctggctctgctctccgaggccgtgctgagagggcaggccctgctggtcaactctagccagccctgggagcccctgcagctgcacgtggacaaggccgtgagcggcctgagaagcctgaccaccctgctgagagccctgggcgcacagaaagaggccatcagccccccagacgccgcctctgccgccccactgaggaccatcaccgccgacaccttcaggaagctgttcagggtctacagcaacttcctgaggggcaagctgaagctgtacaccggcgaggcctgcaggaccggcgacagatgatgagcggccgcgactctagcggccgc

SEQ ID NO: 8 - DNA, 900 bp, Artificial Sequence
  partially synthesized
gtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaattaatacgactcactatagggagacagactgttcctttcctgggtcttttctgcaggcaccgtcgtcgacttaacagatctcgagctcaagcttcgacgagattttcaggagactaaggaagctaaaatgggcgtgcacgagtgccccgcctggctgtggctgctgctgtccctgctgtctctgcccctgggcctgccagtgctgggagccccacccaggctgatctgcgacagcagggtgctggaaagatacctgctggaagccaaagaggccgagaacatcaccaccggctgcaacgagacatgcagcctgaacgagaacattacagtgcccgacaccaaggtgaacttctacgcctggaagaggatggaagtcggccagcaggccgtggaggtctggcagggcctggctctgctctccgaggccgtgctgagagggcaggccctgctggtcaactctagccaggtcaacgagactctgcagctgcacgtggacaaggccgtgagcggcctgagaagcctgaccaccctgctgagagccctgggcgcacagaaagaggccatcagccccccagacgccgcctctgccgccccactgaggaccatcaccgccgacaccttcaggaagctgttcagggtctacagcaacttcctgaggggcaagctgaagctgtacaccggcgaggcctgcaggaccggcgacagatgatga

SEQ ID NO: 9 - DNA, 915 bp, Artificial Sequence
  partially synthesized
gtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagactctataggcacacccctttggctcttatgcatgaattaatacgactcactatagggagacagactgttcctttcctgggtcttttctgcaggcaccgtcgtcgacttaacagatctcgagctcaagcttcgacgagattttcaggagactaaggaagctaaaatgggcgtgcacgagtgccccgcctggctgtggctgctgctgtccctgctgtctctgcccctgggcctgccagtgctgggagccccacccaggctgatctgcgacagcagggtgctggaaagatacctgctggaagccaaagaggccgagaacatcaccaccggctgcgccgagcactgcagcctgaacgagaacattacagtgcccgacaccaaggtgaacttctacgcctggaagaggatggaagtcggccagcaggccgtggaggtctggcagggcctggctctgctctccgaggccgtgctgagagggcaggccctgctggtcaactctagccagccctgggagcccctgcagctgcacgtggacaaggccgtgagcggcctgagaagcctgaccaccctgctgagagccctgggcgcacagaaagaggccatcagccccccagacgccgcctctgccgccccactgaggaccatcaccgccgacaccttcaggaagctgttcagggtctacagcaacttcctgaggggcaagctgaagctgtacaccggcgaggcctgcaggaccggcgacagatgatgagcggccgcgactcta